(19) United States
(12) Patent Application Publication
(10) Pub. No.: US 2014/0170922 A1
(43) Pub. Date: Jun. 19, 2014

(54) Low Density Fibers and Methods for Forming Same

(71) Applicant: Kimberly-Clark Worldwide, Inc., Neenah, WI (US)

(72) Inventors: Simon K. Poruthoor, Alpharetta, GA (US); Charles W. Colman, Marietta, GA (US); Jeffrey J. Krueger, Rowell, GA (US); Ryan J. McEneany, Appleton, WI (US); Bryan D. Haynes, Cummings, GA (US); Wing-Chak Richard Ng, Suwanee, GA (US)

(73) Assignee: Kimberly-Clark Worldwide, Inc., Neenah, WI (US)

(21) Appl. No.: 14/077,637

(22) Filed: Nov. 12, 2013

Related U.S. Application Data
(60) Provisional application No. 61/739,421, filed on Dec. 19, 2012.

(51) Int. Cl. C08K 7/26; B29C 44/20; B29C 55/30; B29C 47/00; A61L 15/18; A61L 15/24

(52) U.S. Cl. CPC C08K 7/26; A61L 15/18; A61L 15/24; B29C 55/30; B29C 47/0014; B29C 44/20
USPC 442/327; 428/401; 521/143; 521/182; 264/54

(57) ABSTRACT

Fibers that are formed from a thermoplastic composition that contains a polymer and high surface area nanostructures are provided. The fibers have a voided structure and low density while maintaining good strength characteristics. To achieve such a structure, a blowing agent in the thermoplastic composition is activated during extrusion to form bubbles in the fibers. The high surface area nanostructures in the formed fibers can be formed of or carry the blowing agent and can enhance the strength of the fibers and compensate for the non-load bearing voids of the fibers.

### PRIORITY

This application claims the benefit of Provisional Application Ser. No. 61/739,421 filed on Dec. 19, 2012, the entire disclosure of which is incorporated herein by reference.

### BACKGROUND OF THE INVENTION

Various attempts have been made to form polymeric fibers while more efficiently utilizing raw materials, particularly raw materials developed from non-renewable resources. In one attempt, fibers have been formed to include a variety of fillers and/or polymers developed from renewable resources. Unfortunately, such fibers have merely replaced one material for another, often with the replacement coming at higher manufacturing cost and often with a loss in desirable fiber characteristics. In another approach, gaseous blowing agents have been employed to help create a cellular “foamed” structure having an amount of voids and reducing the total amount of materials needed to form the fibers. Unfortunately, it has proven very difficult to utilize this technique in forming fibers, particularly in small fibers, as it has proven difficult to control the size of the gaseous bubbles formed in the polymeric compositions because once formed, the bubbles tend to aggregate and/or diffuse out of the polymeric composition, particularly during formation operations such as drawing. In addition, forming open voids throughout the fibers has often proven detrimental to desired characteristics of the formed fibers such as tensile strength and tensile modulus.

As such, a need currently exists for polymeric fibers that utilize fewer materials that can also demonstrate good mechanical properties.

### SUMMARY OF THE INVENTION

In accordance with one embodiment of the present invention, a low density fiber is disclosed that is formed from a thermoplastic composition. The thermoplastic composition comprises at least one polymer and high surface area nanostructures. For instance, the fiber includes from about 0.5 wt. % to about 4 wt. % of the high surface area nanostructures. The fiber includes a plurality of voids that are dispersed within the fiber. The fiber has a density that is about 95% or less of the density of the polymer, and the average percent volume of the fiber that is occupied by the voids is from about 10% to about 50% of the fiber.

In accordance with another embodiment of the present invention, a method for forming a low density drawn fiber is disclosed that comprises loading high surface area nanostructures with a blowing agent; forming a blend that contains a polymer and the high surface area nanostructures carrying the blowing agent; extruding the blend through an extrusion process and a die at a temperature at which a blowing agent in the blend decomposes or reacts to form bubbles in the blend; and drawing the extrusion product to form a drawn fiber that contains a plurality of voids and a plurality of high surface area nanostructures. The fiber can have a density that is about 95% or less of the density of the polymer, and the average percent volume of the fiber that is occupied by the voids can be from about 10% to about 50% of the fiber.

In another embodiment, a method is disclosed in which the high surface area nanostructures are formed of the blowing agent. According to this embodiment, the blend can be extruded at a temperature at which the blowing agent decomposes or reacts to form bubbles and product high surface area nanostructures.

In accordance with yet another embodiment of the present invention, a method for forming a nonwoven web is disclosed that comprises randomly depositing a plurality of fibers onto a forming surface. The fibers may be formed from a blend, such as described herein. The method further comprises drawing the fibers, wherein the fibers contain a plurality of high surface area nanostructures and a plurality of voids.

Other features and aspects of the present invention are discussed in greater detail below.

### BRIEF DESCRIPTION OF THE FIGURES

A full and enabling disclosure of the present invention, including the best mode thereof, directed to one of ordinary skill in the art, is set forth more particularly in the remainder of the specification, which makes reference to the appended figures in which:

FIG. 1 is a schematic illustration of a process that may be used in one embodiment of the present invention to form fibers; and

FIG. 2 is a scanning electron microscope (SEM) image of a cross-section of a drawn fiber as described herein.

FIG. 3 is an SEM image of a cross-section of another drawn fiber as described herein.

FIG. 4 is an SEM image of a cross-section of another drawn fiber as described herein.

FIG. 5 is an optical microscope image of a top view of an undrawn fiber as described herein.

FIG. 6 is an optical microscope image of a top view of another undrawn fiber as described herein.

FIG. 7 is an optical microscope image of a top view of drawn fibers as described herein.

FIG. 8 is an optical microscope image of a top view of drawn fibers as described herein.

### DETAILED DESCRIPTION OF REPRESENTATIVE EMBODIMENTS

Reference now will be made in detail to various embodiments of the subject matter, one or more examples of which are set forth below. Each example is provided by way of explanation of the subject matter, not limitation of the subject matter. In fact, it will be apparent to those skilled in the art that various modifications and variations may be made in the present disclosure without departing from the scope or spirit of the subject matter. For instance, features illustrated or described as part of one embodiment, may be used on another embodiment to yield a still further embodiment. Thus, it is intended that the present disclosure covers such modifications and variations as come within the scope of the appended claims and their equivalents. In addition, all referenced patent and published patent applications are incorporated herein in their entirety by reference thereto for all purposes.

### DEFINITIONS

As used herein, the term “fibers” refer to elongated extrudates formed by passing a polymer through a forming orifice such as a die. Unless noted otherwise, the term “fibers” includes both discontinuous fibers having a definite length and substantially continuous filaments. Substantially filaments may, for instance, have a length much greater than their diameter, such as a length to diameter ratio (“aspect ratio”) greater than about 15,000 to 1, and in some cases, greater than about 50,000 to 1. In addition, fibers can define a hollow core running longitudinally along the axial length of the fiber.

As used herein, the term “nanostructure” refers to a structure that has at least one dimension on a nanometer scale. In particular, while the nanostructures may, in certain embodiments, have one or more dimensions of greater than about 1000 nanometers, they will define at least one dimension or feature on a nanometer scale, i.e., less than about 1000 nanometers, for instance less than about 500 nanometers, less than about 300 nanometers, or less than about 100 nanometers. For example, a nanostructure can have a length of about 1 micrometer or greater and can have an average width (or diameter) of from about 1 to about 200 nanometers, from about 10 to about 150 nanometers, or from about 25 to about 100 nanometers. In another example, a nanostructure can have one or more dimensions formed on a micrometer scale and can include nano-sized features at a surface of the structure. For example, a nanostructure can include nano-sized (i.e., less than 1000 nanometers in at least one dimension) fibrils or other structures on the surface of the structure, and the base structure can be larger, having a dimension that is greater than 1000 nanometers.

As utilized herein, the term “high surface area nanostructures” refers to nanostructures that include internal and/or external features that increase the total surface area of the structure as compared to a solid structure of the same overall dimensions. For instance, a high surface area nanostructure can include a plurality of relatively small pores throughout all or a portion of the structure that can be interconnected or isolated, can include a relatively large hollow cavity (either open to the surface of the nanostructure or enclosed) within the nanostructure, and/or can include surface features that increase the surface area of the structure as compared to a solid structure of the same overall dimensions and lacking the internal and/or external features.

As used herein, the term “nonwoven web” refers to a web having a structure of individual fibers that are randomly interlaid, not in an identifiable manner as in a knitted or woven fabric. Nonwoven webs include, for example, meltblown webs, spunbond webs, carded webs, wet-laid webs, airlaid webs, coform webs, hydraulically entangled webs, etc. The basis weight of the nonwoven web may generally vary, but is typically from about 5 grams per square meter (“gsm”) to 200 gsm, in some embodiments from about 10 gsm to about 150 gsm, and in some embodiments, from about 15 gsm to about 100 gsm.

As used herein, the term “meltblown” web or layer generally refers to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. Nos. 3,849,241 to Butin, et al.; 4,307,143 to Meitner, et al.; and 4,707,398 to Boggs. Meltblown fibers may be substantially continuous or discontinuous, and are generally tacky when deposited onto a collecting surface.

As used herein, the term “spunbond” web or layer generally refers to a nonwoven web containing small diameter substantially continuous filaments. The filaments are formed by extruding a molten thermoplastic material from a plurality of fine, usually circular, capillaries of a spinnerette with the diameter of the extruded filaments then being rapidly reduced as by, for example, eductive drawing and/or other well-known spunbonding mechanisms. The production of spunbond webs is described and illustrated, for example, in U.S. Pat. Nos. 4,340,563 to Appel, et al., 3,692,618 to Dorschner, et al., 3,802,817 to Matsuki, et al., 3,338,992 to Kinney, 3,341,394 to Kinney, 3,502,763 to Hartmann, 3,502,538 to Petersen, 3,542,615 to Dobo, et al., and 5,382,400 to Pike, et al. Spunbond filaments are generally not tacky when they are deposited onto a collecting surface. Spunbond filaments may sometimes have diameters less than about 40 micrometers, and are often between about 5 to about 20 micrometers.

### DETAILED DESCRIPTION

Generally speaking, the present invention is directed to fibers that are formed from a thermoplastic composition and have a voided structure and low density. To achieve such a structure, a polymer is blended with high surface area nanostructures. During the fiber formation process, a blowing agent that is included in the blend can react to form bubbles that form voids in the fibers. The blowing agent can be provided as a separate component to the blend that can be carried on and/or in the high surface area nanostructures or can be provided via the material that forms the high surface area nanostructures (i.e., active nanostructures). For instance, the blend can include active high surface area nanostructures formed of a blowing agent that can react or decompose at an extrusion temperature to release a gas and form bubbles in the nascent fibers. Following release of the gas from the active high surface area nanostructures, non-volatile by-product of the active nanostructures can remain in the fibers in the form of product high surface area nanostructures. In another embodiment, the high surface area nanostructures of the blend can be inert and the nanostructures of the drawn fibers can be chemically identical to the nanostructures added to the pre-extrusion blend. In this embodiment the blowing agent can be provided as a separate component, as a coating on the surface of the high surface area nanostructures, and/or sequestered within the high surface area nanostructures.

Following initial extrusion, the formed fibers can be stretched or drawn. Without intending to be limited by theory, it is believed that the high surface area nanostructures can provide capillary forces within the extrudate that can reduce gas diffusion, disperse bubbles in the nascent fibers and reduce bubble coalition and diffusion of the bubbles out of the fibers. This creates a plurality of voids (e.g., micro-voids, nano-voids, or a combination thereof) located throughout the fiber. In addition, the high surface area nanostructures in the drawn fibers can enhance the strength of the fibers and compensate for the non-load bearing voids within the fibers.

The presence of the nanostructures in the fibers can provide additional benefits as well. For instance, the nanostructures can provide a route to formation of a hierarchical void structure in the polymer matrix of the fibers due to the different levels of bubble nucleation throughout the fiber as well as due to the reduction of bubble coalescence during formation. In addition, the nanostructures can stabilize the bubbles once formed leading to the formation of finer scale voids near the nanostructures in the drawn fiber. Moreover, the nanostructures can provide a crystalline template in the nascent fibers that can increase the rate of crystal formation in the polymer. This can provide stabilization to the fiber by creating a void-supporting framework and further reducing migration of the bubbles from the polymer matrix during formation and drawing of the fibers.

The average percent volume occupied by the voids within the fibers can be relatively high, such as from about 10% to about 50% of the fibers, in some embodiments from about 15% to about 45% of the fibers, and in some embodiments, from about 20% to about 40% of the fibers.

Such a high void volume significantly lowers the density of the fibers. For example, the density of the fibers can be about 95% or less of the density of the polymer that forms the fibers, for instance from about 50% to about 90% of the polymer density, or from about 60% to about 80% of the polymer density. The density of the fibers is intended to refer to the weight of the fibers divided by the overall volume of the fibers, which would include the volume of the voids within the drawn fibers. The density of the fiber is determined by dividing the weight of the fiber by the total bulk volume of the fiber including the voids in the fiber. In one embodiment, the resulting fibers may have a density of about 0.8 grams per cubic centimeter (“g/cm³”) or less, in some embodiments from about 0.4 g/cm³ to about 0.75 g/cm³, and in some embodiments, from about 0.5 g/cm³ to about 0.7 g/cm³, for instance from about 0.65 g/cm³ to about 0.75 g/cm³. The specific gravity of the fibers can likewise be less than that of the polymer that forms the fibers, for instance about 95% or less, such as from about 50% to about 90% or about 60% to about 80% of the specific gravity of the polymer of the fibers.

The diameter of the fibers may vary depending on the desired application. Typically, the fibers are formed to have a diameter of about 100 micrometers (μm) or less, in some embodiments less than about 50 μm, less than about 25 mm, for instance from about 5 μm to about 20 μm.

Various embodiments of the present invention will now be described in more detail.

### I. Thermoplastic Composition

A. Polymer

One or more polymers typically constitute from about 70 wt. % to about 99 wt. %, in some embodiments from about 75 wt. % to about 98 wt. %, and in some embodiments, from about 80 wt. % to about 95 wt. % of the thermoplastic composition that forms the fibers. A polymer of the thermoplastic composition can possess a relatively high molecular weight that can help improve the melt strength and stability of the thermoplastic composition. The polymer may also have a melt flow rate that can be conducive to fiber formation. The polymer may, for example, have a melt flow rate of from about 0.1 to about 250 grams per 10 minutes, in some embodiments from about 0.5 to about 200 grams per 10 minutes, and in some embodiments, from about 5 to about 150 grams per 10 minutes, determined according to ASTM 01238 at a load of 2160 grams and at 190° C.

The modulus of elasticity of the polymer may generally range from about 2 to about 3000 Megapascals (MPa), in some embodiments from about 5 to about 20000 MPa, and in some embodiments, from about 10 to about 500 MPa.

To impart toughness, the polymer may also exhibit an elongation at break (i.e., the percent elongation of the polymer at its failure point) of about 50% or more, in some embodiments about 100% or more, in some embodiments from about 100% to about 2000%, and in some embodiments, from about 250% to about 1500%. The tensile properties including the modulus of elasticity and the elongation at break may be determined in accordance with ASTM 638-10 at 23° C.

While a wide variety of polymers may be employed that have suitable fiber forming properties such as those identified above, particularly suitable examples of polymers may include, for instance, polyolefins (e.g., polyethylene, polypropylene, polybutylene, etc.); styrenic copolymers (e.g., styrene-butadiene-styrene, styrene-isoprene-styrene, styrene-ethylene-propylene-styrene, styrene-ethylene-butadiene-styrene, etc.); polytetrafluoroethylenes; polyesters (e.g., recycled polyester, polyethylene terephthalate, polylactic acid, etc.); polyvinyl acetates (e.g., poly(ethylene vinyl acetate), polyvinyl chloride acetate, etc.); polyvinyl alcohols (e.g., polyvinyl alcohol, poly(ethylene vinyl alcohol), etc.); polyvinyl butyrals; acrylic resins (e.g., polyacrylate, polymethylacrylate, polymethylmethacrylate, etc.); polyamides (e.g., nylon); polyvinyl chlorides; polyvinylidene chlorides; polystyrenes; polyurethanes; etc. Suitable polyolefins may, for instance, include ethylene polymers (e.g., low density polyethylene (“LDPE”), high density polyethylene (“HDPE”), linear low density polyethylene (“LLDPE”), etc.), propylene homopolymers (e.g., syndiotactic, atactic, isotactic, etc.), propylene copolymers, and so forth.

In one particular embodiment, the polymer is a polyolefin homopolymer or copolymer, such as homopolypropylene or a copolymer of propylene. The propylene polymer may, for instance, be formed from a substantially isotactic polypropylene homopolymer or a copolymer containing equal to or less than about 10 wt. % of other monomer, i.e., at least about 90% by weight propylene. Such homopolymers may have a melting point of from about 160° C. to about 170° C. Suitable propylene homopolymers include those available from ExxonMobil Company of Houston, Tex. such as those available under the designation ACHIEVE™.

In still another embodiment, the polymer may be a copolymer of ethylene or propylene with another α-olefin, such as a C₃-C₂₀ α-olefin or C₃-C₁₂ α-olefin. Specific examples of suitable α-olefins include 1-butene; 3-methyl-1-butene; 3,3-dimethyl-1-butene; 1-pentene; 1-pentene with one or more methyl, ethyl or propyl substituents; 1-hexene with one or more methyl, ethyl or propyl substituents; 1-heptene with one or more methyl, ethyl or propyl substituents; 1-octene with one or more methyl, ethyl or propyl substituents; 1-nonene with one or more methyl, ethyl or propyl substituents; ethyl, methyl or dimethyl-substituted 1-decene; 1-dodecene; and styrene. Particularly desired α-olefin comonomers are 1-butene, 1-hexene and 1-octene. The ethylene or propylene content of such copolymers may be from about 60 mole % to about 99 mole %. In some embodiments from about 80 mole % to about 98.5 mole %, and in some embodiments, from about 87 mole% to about 97.5 mole %. The α-olefin content may likewise range from about 1 mole % to about 40 mole %, in some embodiments from about 1.5 mole % to about 15 mole %, and in some embodiments, from about 2.5 mole % to about 13 mole %.

Exemplary olefin copolymers include ethylene-based copolymers available under the designation EXACT™ from ExxonMobil Chemical Company of Houston, Tex. Other suitable ethylene copolymers are available under the designation ENGAGE™, AFFINITY™, DOWLEX™ (LLDPE) and ATTANE™ (ULDPE) from Dow Chemical Company of Midland, Mich. Other suitable ethylene polymers are described in U.S. Pat. Nos. 4,937,299 to Ewen et al.; 5,218,071 to Tsutsui et al.; 5,272,236 to Lai, et al.; and 5,278,272 to Lai, at al. Suitable propylene copolymers are also commercially available under the designations VISTAMAXX™ from ExxonMobil Chemical Co. of Houston, Tex.; FINA™ (e.g., 8573) from Atofina Chemicals of Feluy, Belgium; TAFMER™ available from Mitsui Petrochemical Industries; and VERSIFY™ available from Dow Chemical Co. of Midland, Mich. Other examples of suitable propylene polymers are described in U.S. Pat. Nos. 6,500,563 to Datta, et al.; 5,539,056 to Yang, et al.; and 5,596,052 to Resconi, et al.

Any of a variety of known techniques may generally be employed to form the olefin copolymers. For instance, olefin polymers may be formed using a free radical or a coordination catalyst (e.g., Ziegler-Natta). Preferably, the olefin polymer is formed from a single-site coordination catalyst, such as a metallocene catalyst. Such a catalyst system produces copolymers in which the comonomer is randomly distributed within a molecular chain and uniformly distributed across the different molecular weight fractions. Metallocene-catalyzed polyolefins are described, for instance, in U.S. Pat. Nos. 5,571,619 to McAlpin et al.; 5,322,728 to Davis et al.; 5,472,775 to Obijeski et al.; 5,272,236 to Lai et al.; and 6,090,325 to Wheat, et al. Examples of metallocene catalysts include bis(n-butylcyclopentadienyl)titanium dichloride, bis(n-butylcyclopentadienyl)zirconium dichloride, bis(cyclopentadienyl)scandium chloride, bis(indenyl)zirconium dichloride, bis(methylcyclopentadienyl)titanium dichloride, bis(methylcyclopentadienyl)zirconium dichloride, cobaltocene, cyclopentadienyltitanium trichloride, ferrocene, hafnocene dichloride, isopropyl(cyclopentadienyl,-1-flourenyl)zirconium dichloride, molybdocene dichloride, nickelocene, niobocene dichloride, ruthenocene, titanocene dichloride, zirconocene chloride hydride, zirconocene dichloride, and so forth. Polymers made using metallocene catalysts typically have a narrow molecular weight range. For instance, metallocene-catalyzed polymers may have polydispersity numbers (M_{w}/Mₙ) of below 4, controlled short chain branching distribution, and controlled isotacticity.

In one embodiment, the polymer may be a polyester homopolymer or copolymer. For instance, the polymer may be an aliphatic polyester such as polylactic acid, polybutylene succinate, etc. One suitable polyester is polylactic acid, which may generally be derived from monomer units of any isomer of lactic acid, such as levorotory-lactic acid (“L-lactic acid”), dextrorotatory-lactic acid (“D-lactic acid”), meso-lactic acid, or mixtures thereof. Monomer units may also be formed from anhydrides of any isomer of lactic acid, including L-lactide, D-lactide, meso-lactide, or mixtures thereof. Cyclic dimers of such lactic acids and/or lactides may also be employed. Any known polymerization method, such as polycondensation or ring-opening polymerization, may be used to polymerize lactic acid. A small amount of a chain-extending agent (e.g., a diisocyanate compound, an epoxy compound or an acid anhydride) may also be employed. The polylactic acid may be a homopolymer or a copolymer, such as one that contains monomer units derived from L-lactic acid and monomer units derived from D-lactic acid. Although not required, the rate of content of one of the monomer unit derived from L-lactic acid and the monomer unit derived from D-lactic acid is preferably about 85 mole % or more, in some embodiments about 90 mole % or more, and in some embodiments, about 95 mole % or more. Multiple polylactic acids, each having a different ratio between the monomer unit derived from L-lactic acid and the monomer unit derived from D-lactic acid, may be blended at an arbitrary percentage. Of course, polylactic acid may also be blended with other types of polymers (e.g., polyolefins, polyesters, etc.)

One specific example of a suitable polylactic acid polymer that may be used is commercially available from Biomer, Inc. (of Krailling, Germany) under the name BIOMER™ L9000. Other suitable polylactic acid polymers are commercially available from Natureworks LLC of Minnetonka, Minn. (NATUREWORKS®) or Mitsui Chemical (LACEA™). Still other suitable polylactic acids may be described in U.S. Pat. Nos. 4,797,468; 5,470,944; 5,770,682; 5,821,327; 5,880,254; and 6,326,458, which are incorporated herein in their entirety by reference thereto for all purposes.

A polyester for use as the polymer is not limited to aliphatic polyesters, and a polyester may be an aromatic polyester such as polyethylene terephthalate, polybutylene terephthalate or a copolymer such as an aliphatic-aromatic copolyester.

B. Nanostructures

The fibers can include the high surface area nanostructures in an amount of about 4 wt. % or less, for instance from about 0.3 wt. % to about 3 wt. %, from about 0.5 wt. % to about 2.5 wt. % or from about 1 wt. % to about 2 wt. % by weight of the fiber.

The high surface area nanostructures may define any overall shape. For instance, the high surface area nanostructures may be spherical, fibrillar, or relatively flat, such as a flat platelet shape. Moreover, the high surface area nanostructures can include any external and/or internal features that may increase the surface area of the nanostructures as compared to a solid nanostructure of the same overall size. For instance, a high surface area nanostructure can be a porous structure that may include a plurality of pores throughout all or a portion of the structures. The pores of a high surface area nanostructure can be isolated or interconnected, or a combination of both, and a portion of the pores may be open to an outer surface of the high surface area nanostructure, though this is not a requirement of the structures.

In one embodiment, the high surface area nanostructures can include a hollow cavity that can be partially or completely enclosed within an outer shell. The outer shell may be solid or may be porous, i.e., can include a plurality of connected or isolated small pores throughout the shell, at least a portion of which may be open at an outer surface of the nanostructure. For example, the high surface area nanostructures can include nanotubes or hollow nanospheres that have a hollow cavity within the structure.

As utilized herein, the term “nanotube” generally refers to a hollow cylindrical structure that has an outer cross sectional diameter of less than about 200 nanometers, for instance from about 50 to about 100 nanometers, and a length that can range from about 200 nanometers to about 3 micrometers, for instance from about 500 nanometers to about 2 micrometers. The inner cross sectional diameter of the nanotubes can generally be about 100 nanometers or less, for instance from about 10 nanometers to about 60 nanometers, or from about 20 nanometers to about 40 nanometers. A nanotube shell can be either solid or porous.

A high surface area nanostructure can include surface features that increase the surface area of the structure as compared to a solid structure of the same overall size. For instance, the high surface area nanostructures may include a plurality of identical features formed on the surface or may include different features formed of various sizes, shapes and combinations thereof. A predetermined pattern of features may include a mixture of features having various lengths, diameters, cross-sectional shapes, and/or spacings between the features. For example, the features may be spaced apart in a uniform manner, such as in a rectangular or square grid or in concentric circles. In one embodiment, features may vary with regard to size and/or shape and may form a complex pattern. At least a portion of the features may be formed on a nano-sized scale, e.g., defining a cross-sectional dimension of less than about 500 nanometers (nm), for instance less than about 400 nm, less than about 250 nm, or less than about 100 nm. The cross sectional dimension of the features can generally be greater than about 5 nm, for instance greater than about 10 nm, or greater than about 20 nm. For example, the surface features can define a cross sectional dimension between about 5 nm and about 500 nm, between about 20 nm and about 400 nm, or between about 100 nm and about 300 nm. In cases where the cross sectional dimension of a feature varies as a function of height of the feature, the cross sectional dimension can be determined as an average from the base to the tip of the feature, or as the maximum cross sectional dimension of the feature, for example the cross sectional dimension at the base of a cone-shaped surface feature.

The thermoplastic composition may incorporate a plurality of substantially identical high surface area nanostructures or optionally may incorporate a mixture of different high surface area nanostructures, including a combination of nanostructure formed of different shapes, formed to different lengths, having different types of surface area increasing features, or any combination thereof.

In one embodiment, the high surface area nanostructures added to the blend can be active high surface area nanostructures formed of a blowing agent that can react or decompose at extrusion conditions to form a gaseous/vaporous product in the form of bubbles in the nascent fiber while retaining a product nanostructure in the fibers. For example, and as described in more detail below, an active high surface area nanostructure can be formed of a material that, at a certain activation temperature (which can correspond with an extrusion temperature for the fibers) can decompose to emit a gas or vapor in the form of bubbles. The decomposition of the material does not destroy the high surface area nanostructure, however. Rather, following emission of the bubbles, a product nanostructure remains in the extrudate. The product nanostructure is also a high surface area nanostructure, but differs chemically from the active high surface area nanostructures that were incorporated in the blend due to the loss of the gas in the form of bubbles. Following drawing and cooling, the bubbles can be retained as voids in the drawn fibers.

Examples of the blowing agent of the active high surface area nanostructures can include materials that can release water in the form of water vapor at an extrusion temperature. Such blowing agents include, without limitation, metal salts of Group 1 or 2 of the Periodic Table in which the anion is a phosphate, chromate, sulfate, borate, carbinate, or the like, said salts containing hydrate water. Suitable salts include, for instance, hydrated potassium aluminum sulfate, magnesium sulfate dihydrate, magnesium sulfate heptahydrate, calcium sulfate dihydrate, potassium citrate monohydrate, tricalcium phosphate monohydrate, sodium perborate tetrahydrate, barium acetate monohydrate and barium borate heptahydrate, among others.

Active high surface area nanostructures can also include water-releasing metal hydroxides such as aluminum hydroxides including aluminum trihydrate (ATH), also known as aluminum trihydroxide (Al(OH)₃), and magnesium hydroxide (Mg(OH)₂). The metal hydroxide can decompose during extrusion to release water and leave a metal oxide hydroxide and/or metal oxide nanostructure in the formed fiber. For example, aluminum hydroxide nanostructures can be included in the blend. Aluminum hydroxide decomposes at approximately 200° C. to form aluminum oxide hydroxide and/or aluminum oxide and water. Upon decomposition, the water can form bubbles in the thermoplastic composition and the aluminum oxide hydroxide and/or aluminum oxide can remain in the extrudate in the form of product high surface area nanostructures.

In general, the blowing agent of an active high surface area nanostructure can decompose to release water (at least in substantial amounts) at a temperature above the melting point of the polymer of the thermoplastic composition, such that the blend can be formed without release of the water. For example, the water release temperature of the blowing agent can be about 10° C. or more above the melting point of the polymer, such as about 20° C., about 25° C., or about 30° C. above the melting point of the polymer. The water release temperature of the blowing agent should also be low enough that such temperature is not detrimental to the polymer of the thermoplastic composition. As such, the blowing agent of an active nanostructure can be selected upon choosing the polymer of the thermoplastic composition and upon determining the melting point and the decomposition temperature of the polymer.

The high surface area nanostructures incorporated in the blend need not be active nanostructures. For instance, the high surface area nanostructures of the blend can be utilized to develop the capillary forces within the thermoplastic composition upon foaming that can prevent agglomeration of the bubbles and reduce dissipation of the bubbles out of the fibers during the formation process, and the material forming the high surface area nanostructures can be inert during the formation process. According to this embodiment, the chemical structure of the materials forming the high surface area nanostructures can be identical in the blend and in the formed fibers.

Inert high surface area nanostructures can be formed of one or more particulate additives as are generally known in the art such as nucleating agents (e.g., calcium carbonate) or particulate fillers. Inert nanostructures can be made from organic or inorganic materials, as well has hybrid materials. Materials for forming inert high surface area nanostructures can include, without limitation, carbon, diatomaceous earth, alumina such as activated alumina, and polymers. Organic high surface area nanostructures can be made from polymers that have a melting temperature greater than the extrusion temperature of the thermoplastic composition such as polystyrene or styrene copolymers, nylon or nylon copolymers, acrylic polymers and copolymers including polymethylmethacrylate, and polyacrylonitrile. The inert high surface area nanostructures can include a zeolite, talc, clay, silicate, fused silicon dioxide, glass, ceramic, metals, metal oxides, etc.

Clay minerals may be particularly suitable for use in the present invention. Examples of such clay minerals include, for instance, talc (Mg₃Si₄O₁₀(OH)₂), halloysite (Al₂Si₂O₅(OH)₄), kaolinite (Al₂Si₂O₅(OH)₄), illite ((K,H₃O)(Al,Mg,Fe)₂ (Si,Al)₄O₁₀[(OH)₂,(H₂O)]), montmorillonite (Na,Ca)_{0.33}(Al,Mg)₂Si₄O₁₀(OH)₂.nH₂O), vermiculite ((MgFe,Al)₃(Al,Si)₄O₁₀(OH)₂.4H₂O), palygorskite ((Mg,Al)₂Si₄O₁₀(OH).4(H₂O)), pyrophyllite (Al₂Si₄O₁₀(OH)₂), etc., as well as combinations thereof.

Inert nanostructures can be utilized as carriers for chemical blowing agents that can thereby be incorporated in the blend. For example, a chemical blowing agent can be coated, absorbed, adsorbed or loaded into and/or on a high surface area nanostructure according to a process that can include contacting the high surface area nanostructures with a solution of the chemical blowing agents, so as to incorporate the chemical blowing agent on and/or in the nanostructures. While not wishing to be bound to any particular theory, it is believe that utilization of an inert high surface area nanostructure as a carrier for a chemical blowing agent can provide the chemical blowing agent in very fine particles throughout the thermoplastic composition, which can encourage formation of smaller bubbles in the thermoplastic composition as well as a more homogeneous distribution of the bubbles throughout the thermoplastic composition.

Optionally, the high surface area nanostructures can include additional characteristics that can encourage interaction between the nanostructure and the blowing agent. For instance, the nanostructures can have a surface charge that can encourage charge/charge interaction between the chemical blowing agent and the nanostructures so as to encourage interaction between the two.

In general, inert high surface area nanostructures can be loaded with a chemical blowing agent by contacting the nanostructures with a solution of the blowing agent, which can load the chemical blowing agent into and/or on the surface of the high surface area nanostructures. The high surface area of the nanostructures can also provide a route for high amounts of the blowing agents to be incorporated in the blend. The loaded nanostructures can then be blended into the thermoplastic composition at a temperature below the activation temperature of the blowing agent. During extrusion, the chemical blowing agent can be activated, leading to the formation of bubbles within the extrudate and leaving the inert high surface area nanostructures in the extrudate, where they can encourage bubble retention within the fibers as well as increase strength characteristics of the low density fibers.

Surface treatment of the nanostructures can also influence the foaming process and the physical properties of the low density fibers. For example, the high surface area nanostructures can be treated with a surface coating and/or surface coupling agents. Surface treatment of the nanostructures can include treatments as are generally known for fillers (see, e.g., U.S. Pat. No. 4,525,494 to Andy).

In some embodiments, the nanostructures may be coated with a functionalized block copolymer for improving compatibility with the polymers in the thermoplastic composition. A first block of the copolymer can be selected to promote bonding between the copolymer and the nanostructures. A second block of the copolymer can be selected for compatibility with the polymer in the thermoplastic composition. By way of example, the first block can include monomeric units of a functionalized acrylic monomer and/or a functionalized vinyl monomer and monomeric units of a vinyl monomer, and the second block can include monomeric units of one or more vinyl monomers and monomeric units of the functionalized acrylic monomer and/or the functionalized vinyl monomer from the first block. One block of the copolymer can be polar, hydrophilic, and miscible and compatible with inorganic nanostructures such as clay nanostructures, and the other block of the copolymer can be nonpolar and exhibit increased compatibility with the polymer of the thermoplastic composition. Such coatings are taught in U.S. Patent Application 2008/0200601 to Flores Santos et al.

Coupling agents that can be surface coated on the nanostructures can include, without limitation, organotitanates, organozirconates, organoaluminates, and so forth (e.g., alkoxy-, neo-alkoxy and cycloheteroatom derivatives thereof). Examples of titanates useful in surface coating the nanostructures include monoalkoxy dioctyl pyrophosphato titanate, neoalkoxy dioctyl pyrophosphato titanate, and the acetylacetonate based titanates.

C. Blowing Agents

The thermoplastic composition can generally constitute about 4 wt. % or less of the blowing agent, for instance, from about 0.3 wt. % to about 3 wt. %, or from about 0.5 wt. % to about 2.5 wt. %. Blowing agents can include those that may be presented in the form of active nanostructures as discussed above as well as any chemical blowing agent suitable for foaming thermoplastic compositions. For instance, a chemical blowing agent can be provided in the blend in conjunction with an inert nanostructure. The chemical blowing agent can react or decompose to release water or any other suitable gaseous product at an extrusion temperature to form bubbles in the nascent fibers. In one embodiment, a chemical blowing agent that completely decomposes at an extrusion temperature to produce one or more gaseous products and no non-volatile by-products can be incorporated in the thermoplastic composition in conjunction with an inert nanostructure.

Chemical blowing agents that produce carbon dioxide and water include carbonate and acid containing compositions, referred to herein as carbonate/acid combinations. For example, a chemical blowing agent can include a citric or tartaric acid in combination with a Group 1 metal. In addition, chemical blowing agents that are capable of releasing nitrogen, carbon monoxide, ammonium, or other gases either alone or in combination are encompassed herein.

Chemical blowing agents can include, without limitation, azodicarbonamide and derivatives, hydrazine derivatives (e.g., 4-4 hydroxybis ((benzenesulphonyl)hydrazide), semicarbazides, tetrazoles, benzoxazines, ammonium carbonates and bicarbonates, and/or citric acid in combination with NaHCO₃.

Commercially available examples of chemical blowing agent include Hydrocerol™ (from B.I. Chemicals), Hostatron™ (from Hoechst Celanese), Expandex™ (from Uniroyal), and Activex™ (from Huber).

D. Other Components

The thermoplastic composition can also include additives as are generally known in the art such as plasticizers (e.g., solid or semi-solid polyethylene glycol), Plasticizers may generally be present in the thermoplastic composition an amount of no more than about 1 wt. %, in some embodiments no more than about 0.5 wt. %, and in some embodiments, from about 0.001 wt. % to about 0.2 wt. % of the thermoplastic composition. Further, due to its stress whitening properties, as described in more detail below, the resulting composition may achieve an opaque color (e.g., white) without the need for conventional pigments, such as titanium dioxide. In certain embodiments, for example, pigments may be present in an amount of no more than about 1 wt. %, in some embodiments no more than about 0.5 wt. %, and in some embodiments, from about 0.001 wt. % to about 0.2 wt. % of the thermoplastic composition.

Of course, a wide variety of ingredients may be utilized in the composition for a variety of different reasons. For instance, materials that may be used include, without limitation, catalysts, antioxidants, stabilizers, surfactants, waxes, solid solvents, and other materials added to enhance the processability of the thermoplastic composition.

### II. Blending

The components of the thermoplastic composition may be blended together using any of a variety of known techniques. In one embodiment, for example, the components may be supplied separately or in combination. For instance, the components may first be dry mixed together to form an essentially homogeneous dry mixture, and they may likewise be supplied either simultaneously or in sequence to a melt processing device that dispersively blends the materials. Batch and/or continuous melt processing techniques may be employed. For example, a mixer/kneader, Banbury mixer, Farrel continuous mixer, single-screw extruder, twin-screw extruder, roll mill, etc., may be utilized to blend and melt process the materials. Particularly suitable melt processing devices may be a co-rotating, twin-screw extruder (e.g., ZSK-30 extruder available from Werner & Pfleiderer Corporation of Ramsey, N.J. or a Thermo Prism™ USALAB 16 extruder available from Thermo Electron Corp., Stone, England). Such extruders may include feeding and venting ports and provide high intensity distributive and dispersive mixing. For example, the components may be fed to the same or different feeding ports of the twin-screw extruder and melt blended to form a substantially homogeneous melted mixture. If desired, other additives may also be injected into the polymer melt and/or separately fed into the extruder at a different point along its length.

The degree of shear/pressure and heat may be controlled to ensure sufficient dispersion of the nanostructures throughout the thermoplastic composition, but not so high as to instigate activation of the blowing agent and release of the gaseous product into the melt prior to extrusion. For example, blending typically occurs at a temperature of from about 150° C. to about 180° C., in some embodiments from about 160° C. to about 175° C. Of course, temperature of blending can vary depending upon the activation temperature of the blowing agent in the blend. Likewise, the apparent shear rate during melt processing may range from about 10 seconds⁻¹ to about 3000 seconds⁻¹, in some embodiments from about 50 seconds⁻¹ to about 2000 seconds⁻¹, and in some embodiments, from about 100 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows. Of course, other variables, such as the residence time during melt processing, which is inversely proportional to throughput rate, may also be controlled to achieve the desired degree of homogeneity.

To achieve the desired shear conditions (e.g., rate, residence time, shear rate, melt processing temperature, etc.), the speed of the extruder screw(s) may be selected with a certain range. Generally, an increase in product temperature is observed with increasing screw speed due to the additional mechanical energy input into the system. For example, the screw speed may range from about 50 to about 300 revolutions per minute (“rpm”), in some embodiments from about 70 to about 500 rpm, and in some embodiments, from about 100 to about 300 rpm. This may result in a temperature that is sufficiently high to disperse the nanostructures without activating the blowing agent of the blend. The melt shear rate, and in turn the degree to which the components of the blend are dispersed, may also be increased through the use of one or more distributive and/or dispersive mixing elements within the mixing section of the extruder. Suitable distributive mixers for single screw extruders may include, for instance, Saxon, Dulmage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin (VIP) mixers.

### III. Fiber Formation

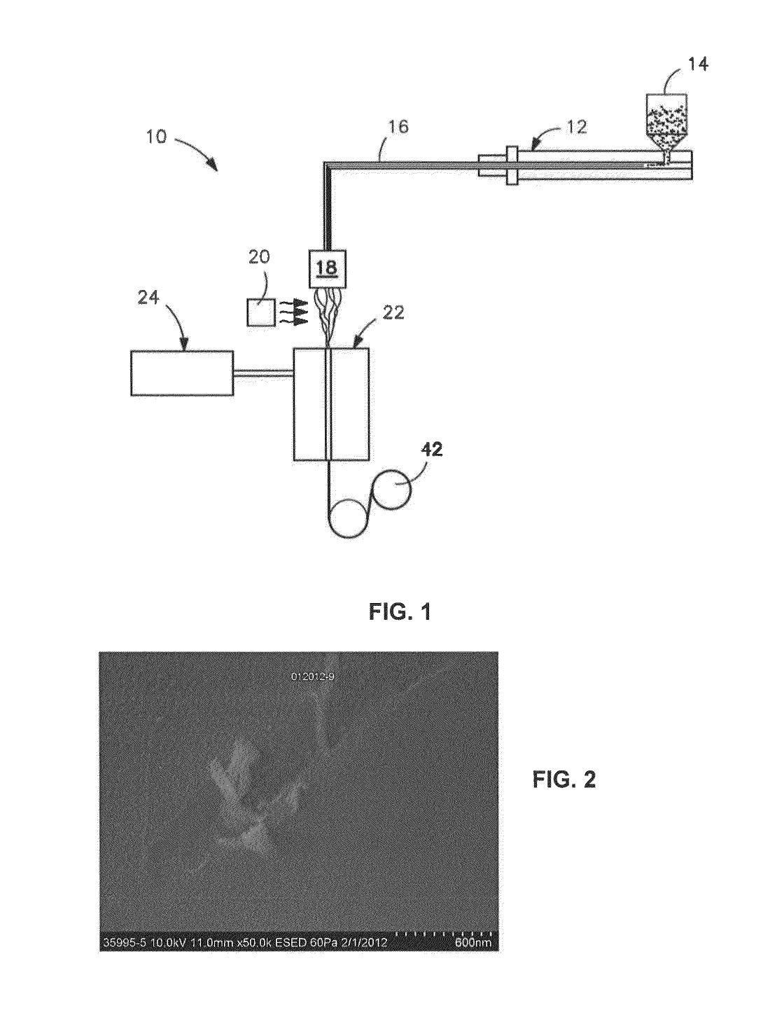

Any of a variety of processes may be used to form fibers in accordance with the present invention. For example, the thermoplastic composition described above may be extruded through a spinneret at a temperature at which the blowing agent is activated and quenched. Referring to FIG. 1, for example, one embodiment of a method for forming fibers is shown in more detail. In this particular embodiment, the thermoplastic composition may be fed into an extruder 12 from a hopper 14. The blend may be provided to the hopper 14 using any conventional technique.

The extruder 12 is heated to a temperature sufficient to extrude the melted polymer and activate the blowing agent, for instance a temperature of between about 180° C. and about 250° C., though, of course, the extrusion temperature will depend upon the specific blowing agent utilized. The extruded composition is then passed through a polymer conduit 16 to a spinneret 18. For example, the spinneret 18 may include a housing containing a spin pack having a plurality of plates stacked one on top of each other and having a pattern of openings arranged to create flow paths for directing polymer components. The spinneret 18 may also have openings arranged in one or more rows. The openings may form a downwardly extruding curtain of filaments when the polymers are extruded therethrough. The process 10 may also employ a quench blower 20 positioned adjacent the curtain of fibers extending from the spinneret 18. Air from the quench air blower 20 quenches the fibers extending from the spinneret 18. The quench air may be directed from one side of the fiber curtain as shown in FIG. 1 or both sides of the fiber curtain.

To form a fiber with the desired length, the quenched fibers are generally melt drawn, such as using a fiber draw unit 22 as shown in FIG. 1. Fiber draw units or aspirators for use in melt spinning polymers are well-known in the art. Suitable fiber draw units for use in the process of the present invention include a linear fiber aspirator of the type shown in U.S. Pat. No. 3,802,817 to Matsuki, et al. The fiber draw unit 22 generally includes an elongated vertical passage through which the fibers are drawn by aspirating air entering from the sides of the passage and flowing downwardly through the passage. A heater or blower 24 supplies aspirating air to the fiber draw unit 22. The aspirating air draws the fibers and ambient air through the fiber draw unit 22. The flow of gas causes the fibers to draw or attenuate which increases the molecular orientation or crystallinity of the polymers forming the fibers.

Once formed, the fibers may be deposited through the outlet opening of the fiber draw unit 22 and onto a godet roll 42. If desired, the fibers collected on the godet roll 42 may optionally be subjected to additional in line processing and/or converting steps (not shown) as will be understood by those skilled in the art. For example, fibers may be collected and thereafter crimped, texturized, and/or and cut to an average fiber length in the range of from about 3 to about 80 millimeters, in some embodiments from about 4 to about 65 millimeters, and in some embodiments, from about 5 to about 50 millimeters to form staple fibers. The staple or continuous fibers may then be incorporated into a nonwoven web as is known in the art, such as bonded carded webs, through-air bonded webs, spunbond webs, meltbond webs, etc.

Regardless of the particular manner in which they are formed, the fibers are typically drawn (e.g., in the machine direction) to a “stretch ratio” of from about 1.1 to about 1000, in some embodiments from about 2 to about 500, and in some embodiments, from about 5 to about 200. The “stretch ratio” may be determined by dividing the length of a drawn fiber by its length before drawing. The draw rate may also vary to help achieve the desired properties, such as within the range of from about 5% to about 1500% per minute of deformation, in some embodiments from about 10% to about 1000% per minute of deformation, and in some embodiments, from about 100% to about 850% per minute of deformation.

Drawing of the fibers may occur in one or multiple stages. In one embodiment, for example, drawing is completed in-line without having to remove the fibers for separate processing. In other cases, however, the fibers may be drawn to a certain extent in-line, and then removed from the fiber forming machinery and subjected to an additional drawing step. Regardless, various drawing techniques may be employed, such as aspiration (e.g., fiber draw units), tensile frame drawing, biaxial drawing, multi-axial drawing, profile drawing, vacuum drawing, etc.

In certain cases, the voids of the drawn fibers may be “micro-voids” in the sense that at least one dimension of such voids has a size of about 1 micrometer or more. For example, such micro-voids may have a dimension in a direction orthogonal to the axial dimension (i.e., transverse or cross-machine direction) that is about 1 micrometer or more, in some embodiments about 1.5 micrometers or more, and in some embodiments, from about 2 micrometers to about 5 micrometers. This may result in an aspect ratio for the micro-voids (the ratio of the axial dimension to the dimension orthogonal to the axial dimension) of from about 0.1 to about 1, in some embodiments from about 0.2 to about 0.9, and in some embodiments, from about 0.3 to about 0.8. Likewise, “nano-voids” may also be present, either alone or in conjunction with the micro-voids. Each dimension of the nano-voids is typically less than about 1 micrometer, and in some embodiments, from about 25 to about 500 nanometers. The voids may have a very high aspect ratio, for instance, the voids can extend in the axial length of the fiber to a length much greater than that of the cross sectional dimension of the voids. For example, the voids can have a length of up to about 1000 times that of the cross sectional dimension, for instance up to about 5000 microns in length.

If desired, the fibers of the present invention may be subjected to one or more additional processing steps, before and/or after drawing. Examples of such processes include, for instance, groove roll stretching, embossing, coating, etc. The fibers may also be surface treated using any of a variety of known techniques to improve its properties. For example, high energy beams (e.g., plasma, x-rays, e-beam, etc.) may be used to remove or reduce any skin layers that form on the fibers, to change the surface polarity, porosity, topography, etc. If desired, such surface treatment may alternatively be used before and/or after cold drawing of the fibers.

The fibers may also be incorporated into a fabric, such as a woven fabric, knit fabric, nonwoven web, etc. For example, the fibers may be formed into a nonwoven web structure by randomly depositing the fibers onto a forming surface (optionally with the aid of a vacuum) and then bonding the resulting web using any known technique. In one embodiment, an endless forming surface may simply be positioned below a fiber aspiration unit that draws the fibers to the desired extent before the web is formed.

Once formed, the nonwoven web may then be bonded using any conventional technique, such as with an adhesive or autogenously (e.g., fusion and/or self-adhesion of the fibers without an applied external adhesive). Autogenous bonding, for instance, may be achieved through contact of the fibers while they are semi-molten or tacky, or simply by blending a tackifying resin and/or solvent with the polymer used to form the fibers. Suitable autogenous bonding techniques may include ultrasonic bonding, thermal bonding, through-air bonding, calendar bonding, and so forth. For example, the web may be further bonded or embossed with a pattern by a thermo-mechanical process in which the web is passed between a heated smooth anvil roll and a heated pattern roll. The pattern roll may have any raised pattern which provides the desired web properties or appearance. Desirably, the pattern roll defines a raised pattern which defines a plurality of bond locations which define a bond area between about 2% and 30% of the total area of the roll. Exemplary bond patterns include, for instance, those described in U.S. Pat. Nos. 3,855,046 to Hansen et al., 5,620,779 to Levy et al., 5,962,112 to Haynes et al., 6.093,665 to Sayovitz et al., as well as U.S. Design Pat. Nos. 428,267 to Romano et al., 390,708 to Brown: 418,305 to Zander, et al.; 384,508 to Zander et al.; 384,819 to Zander, et al.; 358,035 to Zander, et al.; and 315,990 to Blenke, et al. The pressure between the rolls may be from about 90 to about 36000 kilograms per meter. The pressure between the rolls and the temperature of the rolls is balanced to obtain desired web properties or appearance while maintaining cloth like properties. As is well known to those skilled in the art, the temperature and pressure required may vary depending upon many factors including but not limited to, pattern bond area, polymer properties, fiber properties and nonwoven properties.

In addition to spunbond webs, a variety of other nonwoven webs may also be formed from the thermoplastic composition in accordance with the present invention, such as meltblown webs, bonded carded webs, wet-laid webs, airlaid webs, coform webs, hydraulically entangled webs, etc. For example, the thermoplastic composition may be extruded through a plurality of fine die capillaries into a converging high velocity gas (e.g., air) streams that attenuate the fibers to reduce their diameter. Thereafter, the meltblown fibers are carried by the high velocity gas stream and are deposited on a collecting surface to form a web of randomly dispersed meltblown fibers. Alternatively, the polymer may be formed into a carded web by placing bales of fibers formed from the thermoplastic composition into a picker that separates the fibers. Next, the fibers are sent through a combing or carding unit that further breaks apart and aligns the fibers in the machine direction so as to form a machine direction-oriented fibrous nonwoven web. Once formed, the nonwoven web is typically stabilized by one or more known bonding techniques.

If desired, the nonwoven web may also be a composite that contains a combination of the thermoplastic composition fibers and other types of fibers (e.g., staple fibers, filaments, etc.). For example, additional synthetic fibers may be utilized, such as those formed from polyolefins, e.g., polyethylene, polypropylene, polybutylene, and so forth; polytetrafluoroethylene; polyesters, e.g., polyethylene terephthalate and so forth; polyvinyl acetate; polyvinyl chloride acetate; polyvinyl butyral; acrylic resins, e.g., polyacrylate, polymethylacrylate, polymethylmethacrylate, and so forth; polyamides, e.g., nylon; polyvinyl chloride; polyvinylidene chloride; polystyrene; polyvinyl alcohol; polyurethanes; polylactic acid; etc. Some examples of known synthetic fibers include sheath-core bicomponent fibers available from KoSa Inc. of Charlotte, N.C. under the designations T-255 and T-256, both of which use a polyolefin sheath, or T-254, which has a low melt co-polyester sheath. Still other known bicomponent fibers that may be used include those available from the Chisso Corporation of Moriyama, Japan or Fibervisions LLC of Wilmington, Del. Polylactic acid staple fibers may also be employed, such as those commercially available from Far Eastern Textile, Ltd. of Taiwan. The composite may also contain pulp fibers, such as high-average fiber length pulp, low-average fiber length pulp, or mixtures thereof.

Nonwoven composites may be formed using a variety of known techniques. For example, the nonwoven composite may be a “coform material” that contains a mixture or stabilized matrix of the thermoplastic composition fibers and an absorbent material. As an example, coform materials may be made by a process in which at least one meltblown die head is arranged near a chute through which the absorbent materials are added to the web while it is forming. Such absorbent materials may include, but are not limited to, pulp fibers, superabsorbent particles, inorganic and/or organic absorbent materials, treated polymeric staple fibers, and so forth. The relative percentages of the absorbent material may vary over a wide range depending on the desired characteristics of the nonwoven composite. For example, the nonwoven composite may contain from about 1 wt. % to about 60 wt. %, in some embodiments from 5 wt. % to about 50 wt. %, and in some embodiments, from about 10 wt. % to about 40 wt. % thermoplastic composition fibers. The nonwoven composite may likewise contain from about 40 wt. % to about 99 wt. %, in some embodiments from 50 wt. % to about 95 wt. %, and in some embodiments, from about 60 wt. % to about 90 wt. % absorbent material. Some examples of such coform materials are disclosed in U.S. Pat. Nos. 4,100,324 to Anderson, et al.; 5,284,703 to Everhart, et al.; and 5,350,624 to Georger, et al.

Nonwoven laminates may also be formed in which one or more layers are formed from low density fibers of the thermoplastic composition. For example, the nonwoven web of one layer may be a spunbond that contains low density fibers of the thermoplastic composition, while the nonwoven web of another layer contains fibers of the same or other compositions. In one embodiment, the nonwoven laminate contains a meltblown layer positioned between two spunbond layers to form a spunbond/meltblown spunbond (“SMS”) laminate. If desired, fibers of the spunbond layer(s) may be formed from the thermoplastic composition. The meltblown layer may include fibers formed from the thermoplastic composition, and/or any other polymer. Various techniques for forming SMS laminates are described in U.S. Pat. Nos. 4,041,203 to Brock et al,; 5,213,881 to Timmons, et al.; 5,464,688 to Timmons, et al.; 4,374,888 to Bornslaeger; 5,169,706 to Collier, IV, et al.; and 4,766,029 to Brock et al., as well as U.S. Patent Application Publication No. 2004/0002273 to Fitting, et al. Of course, the nonwoven laminate may have other configuration and possess any desired number of meltblown and spunbond layers, such as spunbond/meltblown/meltblown/spunbond laminates (“SIMS”), spunbond/meltblown laminates (“SIM”), etc. Although the basis weight of the nonwoven laminate may be tailored to the desired application, it generally ranges from about 10 to about 300 grams per square meter (“gsm”), in some embodiments from about 25 to about 200 gsm, and in some embodiments, from about 40 to about 150 gsm.

If desired, the fibers, nonwoven web, etc., may also be annealed to help ensure that they retains the desired shape. Annealing typically occurs at temperatures above the glass transition temperature of the polymer, such as at temperatures of from about 65° to about 120° C., in some embodiments from about 70° C. to about 110° C., and in some embodiments, from about 80° C. to about 100° C. The fibers may also be surface treated using any of a variety of known techniques to improve its properties. For example, high energy beams (e.g., plasma, x-rays, e-beam, etc.) may be used to remove or reduce any skin layers that form on the fibers, to change the surface polarity, embrittle a surface layer, etc. If desired, such surface treatment may be used before and/or after formation of a web, as well as before and/or after drawing of the fibers.

### IV. Articles

The fibers and/or a web formed therefrom may be used in a wide variety of applications. For example, the fibers may be incorporated into a “medical product”, such as gowns, surgical drapes, facemasks, head coverings, surgical caps, shoe coverings, sterilization wraps, warming blankets, heating pads, and so forth. Of course, the fibers may also be used in various other articles. For example, the fibers may be incorporated into an “absorbent article” that is capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, mitt wipe, and so forth; medical absorbent articles, such as garments, fenestration materials, underpads, bedpads, bandages, absorbent drapes, and medical wipes; food service wipers; clothing articles; pouches, and so forth. Materials and processes suitable for forming such articles are well known to those skilled in the art. Absorbent articles, for instance, typically include a substantially liquid-impermeable layer (e.g., outer cover), a liquid-permeable layer (e.g., bodyside liner, surge layer, etc.), and an absorbent core. Fibers of the present invention can be used to form a portion or all of any one of the components forming such absorbent articles. In one embodiment, for example, a nonwoven web formed from the fibers of the present invention may be used to form an outer cover of an absorbent article. If desired, the nonwoven web may be laminated to a liquid-impermeable film that is either vapor-permeable or vapor-impermeable.

The present invention may be better understood with reference to the following examples.

### EXAMPLE 1

Halloysite clay nanotubes having an average diameter of about 50-100 nm and lengths which ranged between about 500 to 2000 nm (obtained from Macro-M (Lermo, EDO Mex)) were utilized as nanostructures in a thermoplastic composition. The blowing agent used was Celogen AZ120 (available from Lion Copolymer Geismar, LLC., (LA, USA)) which is an azodicarbonamide blowing agent. The AZ 120 is not soluble in common solvents but it has some solubility in hot water. The AZ 120 powders were dissolved in hot water at about 95° C. The solution was mixed with the clay nanotubes to incorporate the blowing agent inside the hollow core of the nanotubes. The nanotubes were then rinsed and dried.

Following, the dried clay nanotubes loaded with the blowing agent were compounded with metallocene Polypropylene Achieve 6936G1 having a melt flow rate of 1550 g/min (available from Exxon Mobil Chemical Corporation) and the compounding temperature of the extruder was set at 170° C. so that it did not activate the blowing agent. (The activation temperature of AZ120 is about 190°-220° C.). The weight percentage of this compound was—Achieve 6936G1 at 75%, Clay nanotubes at 19% and AZ12O at 6%.

10% weight of this compound was then blended with 90% Polypropylene PP 3155 having a melt flow rate of 36 g/10 min (available from ExxonMobil Chemical Corporation). The thermoplastic composition thus formed was extruded by a spunbond process into fibers. The extruder and die temperatures were at 250° C. to ensure activation of the blowing agent and production of bubbles in the formed fibers. The fibers were collected both with and without drawing. The drawn fibers had a diameter of about 15 to about 25 μm. The fibers were evaluated using SEM and optical microscopes.

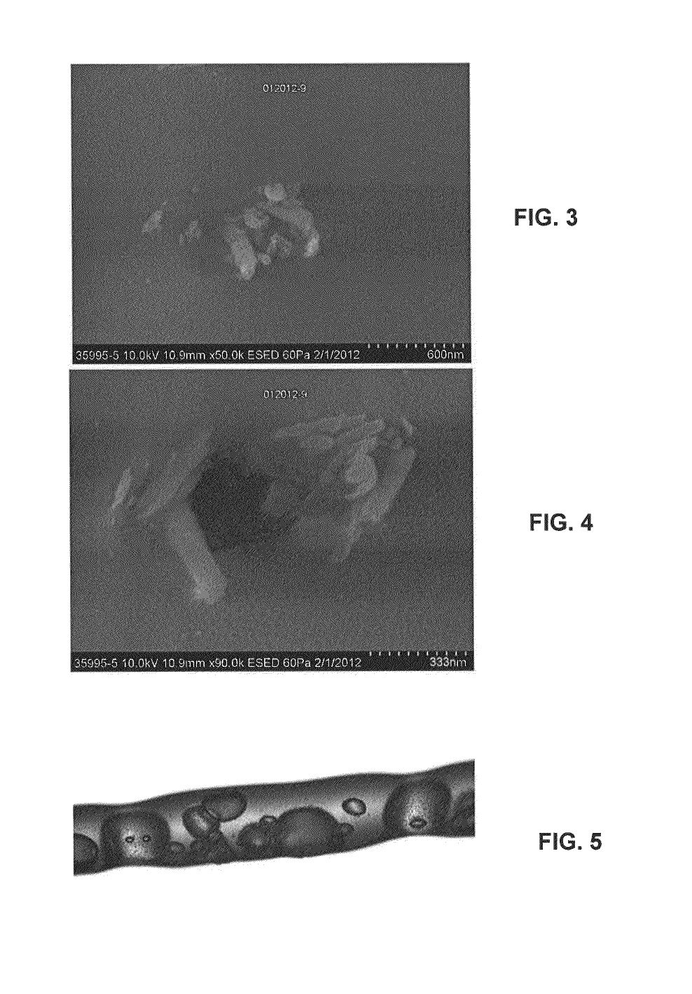

FIGS. 2, 3 and 4 are SEM images showing cross-sections of the drawn fibers. As can be seen, the fibers include voids (or bubbles) adjacent to the nanostructures.

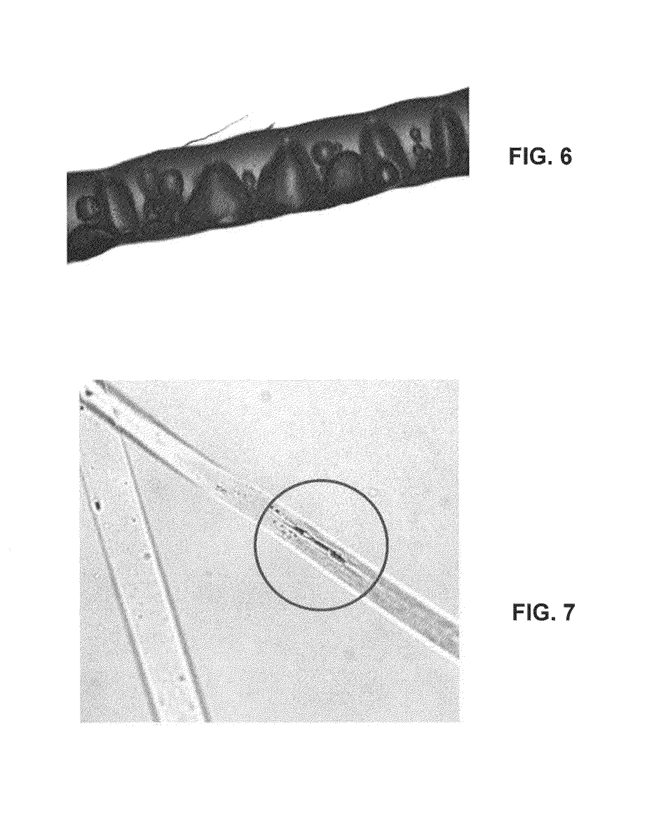

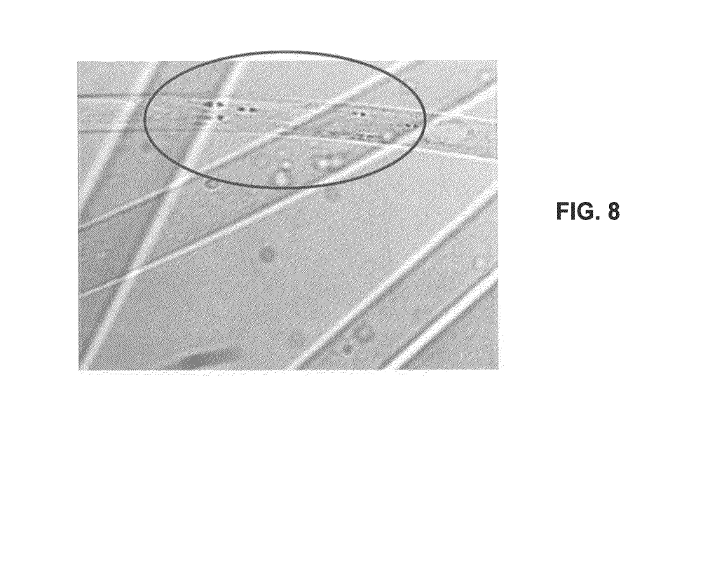

FIGS. 5 and 6 present optical microscope images of undrawn fibers. The gas bubbles are clearly seen in the fibers. FIG. 7 and FIG. 8 are optical microscope images of drawn fibers. As can be seen, the voids are formed throughout the undrawn fibers and are maintained within the fibers in conjunction with the nanostructures following drawing. Thus, the drawn fibers can exhibit low density as well as desirable strength characteristics and can be formed with less raw materials.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A fiber that is formed from a thermoplastic composition, the thermoplastic composition comprising at least one polymer and high surface area nanostructures, the fiber including from about 0.5 wt. % to about 4 wt. % of the high surface area nanostructures based upon the total weight of the fiber, the fiber including a plurality of voids dispersed within the fiber, the fiber having a density that is about 95% or less of the density of the polymer, the average percent volume of the fiber that is occupied by the voids being from about 10% to about 50% of the fiber.

2. The fiber of claim 1, wherein the fiber has a density that is from about 50% to about 90% of the density of the polymer.

3. The fiber of claim 1, wherein the average percent volume of the fiber that is occupied by the voids is from about 15% to about 45% of the fiber.

4. The fiber of claim 1, wherein the voids contain a combination of micro-voids and nano-voids.

5. The fiber of claim 1, wherein the polymer is a polyolefin homopolymer or copolymer.

6. The fiber of claim 5 wherein the polyolefin is a propylene homopolymer, propylene/α-olefin copolymer, ethylene/α-olefin copolymer, or a combination thereof.

7. The fiber of claim 1, wherein the polymer is a polyester homopolymer or copolymer.

8. The fiber of claim 7, wherein the polyester is a polylactic acid or polyethylene terephthalate homopolymer or copolymer.

9. The fiber of claim 1, wherein the fiber has a diameter of about 100 micrometers or less.

10. The fiber of claim 1, wherein the high surface area nanostructures are nanotubes and/or nanospheres.

11. The fiber of claim 1, wherein the high surface area nanostructures comprise a salt.

12. The fiber of claim 11, wherein the salt is a metal salt of Group 1 or Group 2 of the periodic table in which the anion is a phosphate, chromate, sulfate, borate, or carbinate.

13. The fiber of claim 1, wherein the high surface area nanostructures comprise a metal oxide hydroxide and/or a metal oxide.

14. The fiber of claim 13, wherein the metal is aluminum.

15. The fiber of claim 1, wherein the high surface area nanostructures are inert.

16. The fiber of claim 15 wherein the high surface area nanostructures are clay nanostructures.

17. A nonwoven web comprising the fiber of claim 1.

18. An absorbent article comprising an absorbent core positioned between a liquid-permeable layer and a generally liquid-impermeable layer, the absorbent article comprising the nonwoven web of claim 17.

19. A method for forming a low density drawn fiber, the method comprising:
loading high surface area nanostructures with a blowing agent such that the high surface area nanostructures carry the blowing agent;
forming a blend that contains a polymer and the high surface area nanostructures carrying the blowing agent;
extruding the blend through an extrusion process and a die to form a fiber, the extrusion being carried out at a temperature at which the blowing agent decomposes or reacts to form bubbles; and
drawing the fiber, the low density drawn fiber containing a plurality of voids and having a density that is about 95% or less of the density of the polymer, wherein the average percent volume of the drawn fiber that is occupied by the voids is from about 10% to about 50% of the fiber.

20. The method of claim 19, wherein the fiber has a density that is from about 50% to about 90% of the density of the polymer.

21. The method of claim 19, wherein the polymer is a polyolefin homopolymer or copolymer.

22. The method of claim 21, wherein the polyolefin is a propylene homopolymer, propylene/α-olefin copolymer, ethylene/α-olefin copolymer, or a combination thereof.

23. The method of claim 19, wherein the extrusion temperature at which the blowing agent decomposes or reacts is about 10° C. or more above the melting point of the polymer.

24. The method of claim 19, wherein the blend comprises the blowing agent in an amount of about 4 wt. % or less.

25. The method of claim 19, wherein the blend comprises the nanostructures in an amount of about 4 wt. % or less.

26. A method for forming a low density drawn fiber, the method comprising:
forming a blend that contains a polymer and high surface area nanostructures that are formed of a blowing agent;
extruding the blend through an extrusion process and a die to form a fiber, the extrusion being carried out at a temperature at which the blowing agent decomposes or reacts to form bubbles and product high surface area nanostructures; and
drawing the fiber, the low density drawn fiber containing a plurality of voids and having a density that is about 95% or less of the density of the polymer, wherein the average percent volume of the drawn fiber that is occupied by the voids is from about 10% to about 50% of the fiber.

27. The method of claim 26, wherein the fiber has a density that is from about 50% to about 90% of the density of the polymer.

28. The method of claim 26, wherein the polymer is a polyolefin homopolymer or copolymer.

29. The method of claim 28, wherein the polyolefin is a propylene homopolymer, propylene/α-olefin copolymer, ethylene/α-olefin copolymer, or a combination thereof.

30. The method of claim 26, wherein the blowing agent decomposes at the temperature to release water vapor bubbles.

31. The method of claim 26, wherein the extrusion temperature at which the blowing agent decomposes or reacts is about 10° C. or more above the melting point of the polymer.

32. The method of claim 26, wherein the blend comprises the nanostructures in an amount of about 4 wt. % or less.

33. A method for forming a nonwoven web, the method comprising:
forming a blend that contains a polymer and high surface area nanostructures, the high surface area nanostructures carrying a blowing agent;
extruding the blend through a die to form a plurality of fibers, the extrusion being carried out at a temperature at which a blowing agent decomposes or reacts to form bubbles;
drawing the fibers, the drawn fibers containing a plurality of voids and having a density that is about 95% or less of the density of the polymer; and
randomly depositing the drawn fibers onto a surface to form a nonwoven web.

34. A method for forming a nonwoven web, the method comprising:
forming a blend that contains a polymer and high surface area nanostructures, the high surface area nanostructures being formed of a blowing agent;
extruding the blend through a die to form a plurality of fibers, the extrusion being carried out at a temperature at which a blowing agent decomposes or reacts to form bubbles and product high surface area nanostructures;
drawing the fibers, the drawn fibers containing a plurality of voids and having a density that is about 95% or less of the density of the polymer; and
randomly depositing the drawn fibers onto a surface to form a nonwoven web.
